(19) United States
(12) Patent Application Publication
(10) Pub. No.: US 2025/0245559 A1
(43) Pub. Date: Jul. 31, 2025

(54) SYSTEMS AND METHODS FOR INTELLIGENT MODEL TRAINING USING RELEVANT DATA OBJECTS

(71) Applicant: Optum, Inc., Minnetonka, MN (US)

(72) Inventors: George AUSTIN, Maplewood, NJ (US); Jagadish VENKATARAMAN, Menlo Park, CA (US); Fazlolah MOHAGHEGH, Frisco, TX (US); Hamid Reza HASSANZADEH, Suwanee, GA (US); Joel David STREMMEL, Iowa City, IA (US); Ardavan SAEEDI, Jersey City, NJ (US); Gregory D. LYNG, Minneapolis, MN (US); Eran HALPERIN, Santa Monica, CA (US); Zahra Mahmoodzadeh POORNAKI, Laguna Niguel, CA (US)

(21) Appl. No.: 18/428,206

(22) Filed: Jan. 31, 2024

(51) Int. Cl. G06N 20/00; G16H 10/60

(52) U.S. Cl. CPC G06N 20/00; G16H 10/60

(57) ABSTRACT

Systems and methods are described for training and/or using a machine-learning model. A first set of textual data is received. Using a trained machine-learning model that is applied to the first set, a classification of the first set is generated. The trained machine-learning model has been trained based on a subset of textual data that resulted from filtering a set of training textual data. The filtering of the set of training textual data to generate the subset of textual data is based on a comparison between the training textual data and a second set of textual data.

### TECHNICAL FIELD

Various embodiments of this disclosure relate generally to techniques for training a machine-learning model, and, more particularly, to systems and methods for training a machine-learning model using filtered portions of training data.

### BACKGROUND

A wide range of machine learning solutions involve processing of large text input. However, large input size generally increases computing cost and complexity, and does not necessarily lead to more accurate computer predictions. For instance, large text input that includes a subset of text that is irrelevant to a computer prediction may cause the machine learning solution to incorrectly value the subset of text, which may lead to inaccurate computer predictions and/or wasting of compute resources in processing the subset of text. Even a modest increase in input size, when compounded across all samples used to train such a model, can vastly increase the complexity, computing cost, and data resources for training the model.

While techniques have been developed to reduce input size, such as automated summarization models, dimension reduction tools, etc., such resources are generally insular, meaning that they consider only the input data itself, and thus generally represent a trade-off between information loss and input size reduction. Moreover, the information pulled out of an input via such techniques may not align or correspond to a human understanding of decision-making procedure. In an example, the “black-box” nature of a conventional data-driven model may make it impossible or impractical to assess how a particular decision is made and, for example, whether industry or regulatory norms, guidelines, or requirements were followed. In another example, conventional methods generally do not benefit from or adhere to the understanding of operational guidelines and best practices, especially when such external guidelines on decision-making may change over time.

This disclosure is directed to addressing one or more challenges such as the above. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

According to certain aspects of the disclosure, methods and systems are disclosed for reducing data size of training data prior to inputting the training data into a machine-learning model, and, more particularly, to systems and methods for using extrinsic data to target and/or control the reduction of training data and training of a machine-learning model through filtered portions of large training data input.

Conventionally, decisions that are based on large or complicated input data generally require at least some level of manual inspection. While automated tools have been developed to aid in analysis of data, such tools generally have significant drawbacks or limitations. In one example, tools to highlight portions of or summarize data are generally insular, and may not take into account a desired target topic or adhere to external guidelines or regulations. Moreover, as the size of data to be analyzed increased, conventional tools used to parse such data, such as a machine-learning model, generally become more difficult and computationally costly to train and/or use. Often, as the size of data to be analyzed increases, conventional analysis tools exhibit a trade-off between accuracy and computational cost. Further, such tools generally operate as a “black box,” and provide little or no explanation for how or why resulting decisions are made.

Accordingly, improvements in technology relating to automated tools for analyzing large data are needed. As will be discussed in more detail below, in various embodiments, extrinsic guidance, e.g., data and/or criteria separate from training data, is used to guide a process of training a machine-learning model. In an example, guidance data is used to highlight or filter portions from the samples of training data, e.g., to generate a training set that is specifically tailored to the guidance data. As discussed in further detail below, one or more aspects of the disclosure represent improvements to the field of computing. For example, via one or more aspects of the disclosure, a computing efficiency may be increased when generating predictions or decisions for a given level of accuracy and/or a level of accuracy may increase for a given computing cost. In another example, the guidance data may include domain-specific information regarding the training data. Using such domain-specific information to guide training of the machine-learning model may thus provide improved accuracy, precision, and/or efficiency over approaches that attempt to leverage statistical features of the training data (e.g., via dimension reduction or feature engineering).

In some aspects, the techniques described herein relate to a computer-implemented method, including: receiving, by one or more processors, a first set of textual data; and generating, via the one or more processors and using a trained machine-learning model that is applied to the first set of textual data, a classification of the first set of textual data, wherein: the trained machine-learning model has been trained based on a subset of textual data that resulted from filtering a set of training textual data; and the filtering of the set of training textual data to generate the subset of textual data is based on a comparison between the training textual data and a second set of textual data.

In some aspects, the techniques described herein relate to a system, including: at least one memory storing instructions; and at least one processor operatively connected to the at least one memory and configured to execute the instructions to perform operations. The operations include: receiving a first set of textual data; and generating, using a trained machine-learning model that is applied to the first set of textual data, a classification of the first set of textual data, wherein: the trained machine-learning model has been trained based on a subset of textual data that resulted from filtering a set of training textual data; and the filtering of the set of training textual data to generate the subset of textual data is based on a comparison between the training textual data and a second set of textual data.

In some aspects, the techniques described herein relate to a computer-implemented method, including: receiving, via one or more processors, a set of training textual data that includes respective textual data for each of a plurality of entities; receiving, via the one or more processors, criteria data separate from the set of training textual data that defines one or more criteria for at least one classification; extracting a subset of textual data from the set of training textual data by filtering the set of training textual data based on a comparison between the set of training textual data and the criteria data; and training a machine-learning model, via the one or more processors and using the subset of textual data, to generate the at least one classification of input textual data of an entity.

In some aspects, the techniques described herein relate to a computer-implemented method for training a machine-learning model, the method including: obtaining, via one or more processors, condition data for a plurality of entities; obtaining, via the one or more processors, criteria data separate from the condition data that defines one or more criteria for at least one treatment; filtering the condition data, via the one or more processors and based on the one or more criteria, to generate a condition training data set specific to the at least one treatment; and training a machine-learning model, via the one or more processors and using the condition training data set, such that, in response to the machine-learning model receiving input of medical data of an entity, the machine-learning model is configured to generate a decision associated with the at least one treatment for the entity.

In some aspects, the techniques described herein relate to a system for training a machine-learning model that includes: at least one memory storing instructions; and at least one processor operatively connected to the at least one memory and configured to execute the instructions to perform operations. The operations include: obtaining condition data for a plurality of entities; obtaining criteria data separate from the condition data that defines one or more criteria for at least one treatment; filtering the condition data, based on the one or more criteria, to generate a condition training data set specific to the at least one treatment; and training a machine-learning model, using the condition training data set, such that, in response to the machine-learning model receiving input of medical data of an entity, the machine-learning model is configured to generate a decision associated with the at least one treatment for the entity.

In some aspects, the techniques described herein relate to a non-transitory computer-readable medium comprising instructions for training a machine-learning model, the instructions executable by at least one processor to perform operations. The operations include: obtaining condition data for a plurality of entities; obtaining criteria data separate from the condition data that defines one or more criteria for at least one treatment; filtering the condition data, based on the one or more criteria, to generate a condition training data set specific to the at least one treatment; and training a machine-learning model, using the condition training data set, such that, in response to the machine-learning model receiving input of medical data of an entity, the machine-learning model is configured to generate a decision associated with the at least one treatment for the entity.

In some aspects, the techniques described herein relate to a computer-implemented method for generating a decision associated with at least one treatment for an entity, the method including: obtaining, via at least one processor of a user device, entity-specific condition data; generating, via the at least one processor, the decision associated with the at least one treatment for the entity by inputting the entity-specific condition data into a trained machine-learning model that has been trained, based on condition training data set generated by using one or more criteria to filter condition data for a plurality of entities, wherein the decision generated by the trained machine-learning model includes a separate evaluation of one or more metrics corresponding to the one or more criteria; and generating a text output that includes the one or more metrics, the separate evaluation of the one or more metrics, and an overall decision based on a combined evaluation of each metric.

It is to be understood that both the foregoing general description and the following detailed description include examples and are explanatory only and are not restrictive of the disclosed embodiments, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1 depicts an example environment for a training and/or using a machine-learning model, according to one or more embodiments.

FIG. 2 depicts an example embodiment of a method for training a machine-learning model, according to one or more embodiments.

FIG. 3 depicts another example embodiment of a method for using a machine-learning model, according to one or more embodiments.

FIG. 4 depicts another example embodiment of a method for using a machine-learning model, according to one or more embodiments.

FIG. 5 depicts another example embodiment of a method for training a machine-learning model, according to one or more embodiments.

FIG. 6 depicts an example of a computing device, according to one or more embodiments.

### DETAILED DESCRIPTION

Examples in this disclosure are made with reference to different examples of training and use of machine-learning models for a variety of different tasks. However, it should be understood that reference to any particular activity is provided in this disclosure only for convenience and as an example, and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are explanatory only and are not restrictive of the features, as claimed.

In this disclosure, the term “based on” means “based at least in part on.” The singular forms “a,” “an,” and “the” include plural referents unless the context dictates otherwise. The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

The term “provider” generally encompasses an entity or agent thereof involved in providing goods or services to a person, e.g., healthcare to a patient, and encompasses a doctor, nurse, medical team or organization, insurer, or the like. The term “treatment” need not be medical in nature, although medical treatments such as interventions, prescriptions, etc. are contemplated as examples of possible options for inclusion in a treatment. Terms like “treatment,” “decision,” “recommendation,” “classification”, “prediction,” or the like may be used in different embodiments, as the case may be. It should be understood that features of an embodiment pertaining to a “decision” may be similarly applied to a different embodiment pertaining to a “classification,” or the like. Terms like “subject,” “user,” “entity,” “patient,” or the like generally encompasses a person or entity that is considered for or provided with a treatment, e.g., by a provider. Terms like “guidance,” “criteria,” “extrinsic data,” and the like generally encompass information usable to focus understanding of other data in a particular manner, for a particular task, under a particular lens, etc. Terms like “patient medical records,” “condition data,” or the like may be generally understood to include data, e.g., large data, which is evaluated to form a prediction, recommendation, conclusion, or the like. The term “textual data” may encompass any suitable type of text data, such as the foregoing examples.

As used herein, terms such as “large text input,” “large data,” or the like generally encompasses data or information that would result in multiple pages of output, e.g., when printed out or displayed. In an example, “large data” includes, for example, more than 4,000 characters of text, more than 10,000 characters, etc. Conventionally, analysis of such data, even with machine-learning-based solutions, represents a significant cost in terms of computing resources.

In an example, it may be desirable to analyze a patient's health record, e.g., to inform predictions about the patient's condition, decisions about the patient's treatment or care, etc. Over time, a patient's health record may accumulate multiple pages of text data. However, patient medical records includes data beyond text data, such as images, charts, tables, diagrams, etc. In an example, various sources ascribe an average patient medical record to be up to 60 pages of information or more, and between 25 and 80 megabytes of data (e.g., text and images) or more. Medical imaging or other data types such as anatomical models or simulation data may be much larger in size. In some cases, findings or observations regarding such data is included in a patient medical record alongside or instead of the medical imaging itself. Certain types of information can further increase these values, and the average size of a patient medical record has increased over time and is likely to continue to do so.

One or more aspects of this disclosure pertain to generating a classification based on first textual data, e.g., preauthorization recommendations and/or decisions regarding a potential medical treatment of a patient based on the patient's medical record. A system for training a machine-learning model to make such recommendations or decisions, according to one or more aspects of this disclosure, may leverage modern computing technologies to guide the training of the machine-learning model according to second textual data separate from the first textual data, e.g., criteria data separate from training data. Guidance of the training includes, for example, filtering out or highlighting portions of samples of the training data that are relevant to the criteria data. Such operation, in embodiments, enables training the machine-learning model without or with less compromise of computing complexity, model accuracy, etc.

In an example use case, a machine learning model for evaluating textual data such as patient data to make a classification such as a preauthorization recommendation is trained using a training process that benefits from an understanding of what portions of patient data are relevant to that decision, e.g., as informed by second textual data such as an external guidance resource. In one example, clinical guidance generally circulated to medical providers is used to identify portions of patient data relevant to making preauthorization decisions. For instance, a clinical guidance document is usable with a model or algorithm or the like to identify keywords/portions that are indicative of an intervention/preauthorization outcome, and such keywords/portions are usable as a filter that is applied to training textual data, such as patient data records, to generate training data. In some instances, the training textual data and/or the guidance data includes coded entries, e.g., medical coding classifications or the like. The resulting subset of training data in the form of filtered records are then used to train a machine-learning model that receives, for example, patient data as input.

The patient data used as input for the trained machine-learning model, e.g., to generate a recommendation or decision for preauthorization of a medical treatment of the patient, can be unfiltered and/or filtered in different embodiments. As noted above, data of a large size can significantly increase computing cost and complexity when used as training data for a machine-learning model. However, once a machine-learning model has been trained in a guided fashion, according to one or more aspects of this disclosure, e.g., using a filtered training data set, large-sized data is usable as input with the trained machine-learning model with less or no impact to computing cost, complexity, or accuracy. For instance, once the machine-learning model has been trained in a manner benefiting from guidance, the machine-learning model learns to focus in on material from input data that is relevant to the guidance.

As noted above, in some instances, guidance is in the form of keywords or highlighted portions of guidance data, e.g., keywords or portions of a clinical guidance document determined one or more of manually, via a summarizing algorithm, scoring algorithm, matching or comparison algorithm such as cosine similarity, or the like. In some embodiments, because relevant keywords/portions of guidance have been identified, such knowledge is applied or parsed, e.g., in order to gain a better understanding of the decision-making process. In an example, such knowledge is used to generate one or more metrics, sub-classifications, or sub-decisions from the guidance that cumulatively inform the ultimate classification, recommendation or decision generated by the model. In such embodiments, an identified keyword/portion, or grouping thereof, from an input patient record e.g., in the form of a metric or sub-decision, may be separately evaluated. For instance, beyond just a recommendation or decision as to whether to preauthorize a medical treatment, a trained machine-learning model evaluates constituent questions that lead to that ultimate decision, e.g., whether a required prior treatment was attempted, whether the patient has a prerequisite condition, a score for the patient in a metric assessing risk, etc.

Such separate evaluations are usable to provide information regarding reasoning behind the ultimate conclusion of the model, e.g., whether or not to preauthorize a medical treatment. This type of information is generally not available for conventional approaches using machine-learning which operate as a “black-box.” Further, application of medical treatments generally must adhere to a strict code of medical guidelines and best practices. The visibility of the separate evaluations, according to aspects of this disclosure, is usable to assess whether such guidelines and practices were followed when the recommendation or decision for preauthorization was made. Such visibility also provides a readily available decision record that can be used by a provider to facilitate or guide review of a patient's condition without having to consider the patient's entire medical record. In an example, the separate evaluations are incorporated into output from the machine-learning model in the form of a Question-and-Answer (Q&A) evaluation. In some instances, evaluations or metrics are hierarchical, e.g., metrics A-C impact a score for metric D, and an ultimate preauthorization decision is based on metrics D and E. It should be understood that the foregoing example is illustrative only, and that any suitable relationship between metrics or the like are usable.

In some embodiments, multiple different guidance documents are used to generate a more generalized filter that is applicable to a variety of different medical treatments and/or circumstances. In an example, each different medical treatment is associated with a different metric or hierarchy of metrics, such that a trained machine-learning model is configured to generate a separate evaluation for each.

In some instances, clinical guidance may change from time to time. For example, new medications, medical tools, best practices, etc., may update or replace previous clinical guidance for a medical treatment. In some embodiments, an automated system is configured to monitor and/or periodically pull updated guidance, and configure and instantiate new trained machine-learning models on an ongoing basis, e.g., so that up-to-date models are readily available to evaluate new patient data.

In some embodiments, such as in one or more of the examples discussed above, the guidance data is applied to training data before it is input into the machine-learning model during training. In some embodiments, the guidance data is integrated into the training process. For instance, a guidance document and/or identified keywords/portions are fed as additional input to the machine learning model during training and/or during evaluation of a new patient record. In an example, a generalized machine-learning model that integrates guidance into training is configured to make recommendations and/or decisions regarding a variety of medical treatments. Patient medical records for a plurality of patients that received different medical conditions are used as a basis for generating training data. For each patient, a separate training sample is assembled by combining the patient's medical record with the clinical guidance for the medical treatment that was considered for the patient. In some instances, the patient's medical record is a filtered record generated by applying one or more of the filtering processes discussed above. Additionally, a separate result sample is assembled for each patient that includes, for example, whether or not the medical treatment was approved for the patient, and/or the metrics or sub-evaluations that were used to make that ultimate conclusion.

The collected training samples and the corresponding results samples are then used to train the machine-learning model. Once trained, a resulting machine-learning model is configured to receive, as input, a patient's medical record along with a clinical guidance document corresponding to a particular medical treatment that is being considered for the patient. Further, the resulting machine-learning model is configured to generate, as output, a classification, recommendation, and/or decision as to whether to preauthorize that medical treatment for that patient and/or separate evaluations or metrics which inform that ultimate conclusion.

While several of the examples above involve classifications, recommendations, or decisions for preauthorization of medical treatments, it should be understood that techniques according to this disclosure are adaptable to any suitable type of activity involving analyzing and/or drawing conclusions from data. In an example, one or more aspects of this disclosure may be adapted to prequalification for a loan or mortgage, financial planning, legal analysis, manufacturing or process engineering, construction, building, or site code compliance, etc. It should also be understood that the examples above are illustrative only.

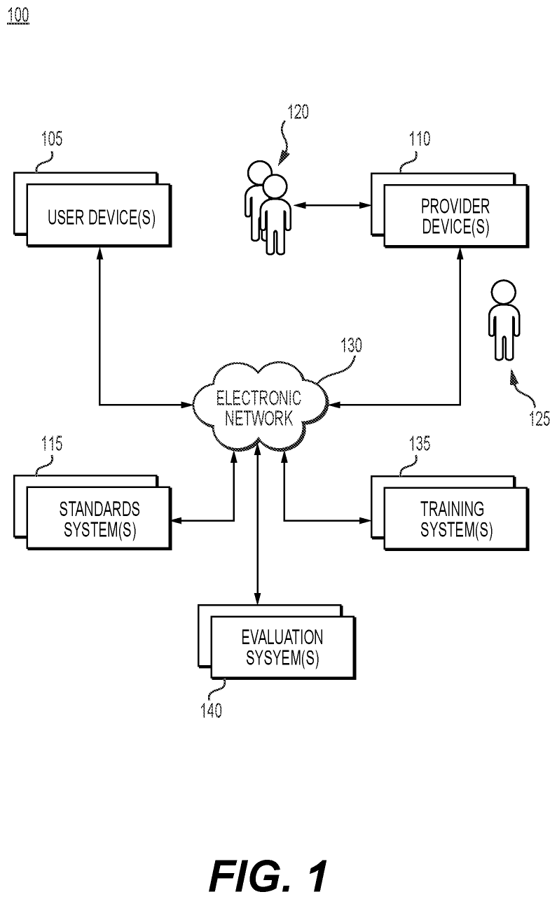

Presented below are various aspects of implementation of training a machine-learning model using external guidance such as criteria data, according to various embodiments. FIG. 1 depicts an example environment 100 that is utilized with training a machine-learning model according to one or more of the techniques presented herein. One or more user device(s) 105, one or more provider device(s) 110, and one or more standards system(s) 115 are configured to communicate across an electronic network 130. As will be discussed in further detail below, one or more training system(s) 135 and one or more evaluation system(s) 140 are configured to communicate with each other and/or one or more of the other components of the environment 100 across the electronic network 130. The one or more provider device(s) 110 is associated with a subject 120, e.g., a patient being considered for one or more medical treatment, e.g., to be provided by a provider 125.

In some embodiments, one or more of the components of the environment 100 are associated with a common entity, e.g., an insurance provider, a medical care provider such as a hospital, a commercial entity, and advertiser, or the like. In some embodiments, one or more of the components of the environment 100 is associated with a different entity than another. The systems and devices of the environment 100 are configured to communicate in any arrangement. As will be discussed herein, systems and/or devices of the environment 100 are configured to communicate in order to one or more of provide medical treatments and/or recommendations or decisions regarding medical treatments to subjects 120, generate, provide, or use machine-learning models for making such recommendations or decisions, storing, accessing, or obtaining patient medical records of subjects 120, storing guidance data such as clinical guidance documents, parsing such guidance data, parsing or filtering the patient medical records to generate training data, and/or storing such training data, among other activities.

The user device 105 is configured to enable a user to access and/or interact with other systems in the environment 100. For example, the user device 105 is a computer system such as, for example, a desktop computer, a mobile device, a tablet, etc. In some embodiments, the user device 105 includes one or more electronic application(s), e.g., a program, browser, etc., installed on a memory of the user device 105. In some embodiments, the electronic application(s) is associated with or enable a user to interact with one or more of the other components in the environment 100. For example, the electronic application(s) includes a browser or application configured to obtain condition data of a subject 120, e.g., patient medical records, obtain guidance data such a criteria data for the preauthorization or performance of a medical treatment, apply guidance data to condition data to generate a training data sample, apply training data samples to train a machine-learning model, and/or apply condition data of a subject 120 to a trained machine-learning model to generate a recommendation or decision.

The provider device 110 includes, for example, a medical data storage system for generating, modifying, storing, and providing access to condition data such as patient medical records. In some embodiments, the provider device 110 is in communication with medical equipment, third party systems, or the like to obtain clinical data for a subject 120, e.g., test results, medical imaging, medical history data, prescription data, etc. In some instances, a provider 125 accesses the provider device 110 via a user device 105, via a portal, an Application Programming Interface (API), or the like, e.g., to enter, modify, or access patient medical data. In some embodiments, condition data includes information regarding prior decisions. For example, patient medical records sometimes include information regarding a patient's condition that was used to decide whether to apply a particular treatment as well as information regarding a decision whether or not such treatment was actually applied. In some instances, condition data includes an evaluation of such prior decisions. For example, a patient medical record sometimes includes an evaluation from after a decision regarding application of a particular treatment was made as to whether the decision was correct (e.g., whether an applied treatment should not have been applied, or whether a denied treatment should have been applied). In some instances, condition data may not include explicit findings regarding the above, but does include post-decision condition data indicative of or usable to determine such findings.

It should be understood that, in some instances, patient medical records may include a plurality of data files stored in different systems, and in different formats. In some instances, the provider device 110 or the like may assemble and/or standardize such data files. However, in some instances, a patient medical record may include data in different formats and/or data stored in different systems. In some instances, a patient medical record includes coded entries, e.g., according to medical coding standards.

The standards system 115, in various embodiments, obtains and/or stores guidance data such as clinical guidance for one or more medical procedures provided by the provider 125. As a general example, the training system 135 and/or evaluation system 140 may be used in conjunction with a model for making a domain-specific classification, e.g., a preauthorization of a particular treatment. The guidance data may include, for example, domain-specific information associated with the domain-specific classification. In an example, an oversight board or authoritative body may, from time to time, publish or issue documents, articles, metrics, criteria, or the like, that indicate one or more standards and practices for providing medical care. In an example, a clinical guidance document may describe the criteria for diagnosing a particular condition, the criteria for which medical treatments may be provided for that condition, as well as how, when, where, with what equipment, etc. A clinical guidance document may include metrics, e.g., a process by which a circumstance is scored, thresholds, recommendations, or the like, as well as observations, procedures, and/or other relevant information. In some instances, the clinical guidance document includes coded entries, e.g., according to medical coding standards. In some instances, the standards system 115 is an online resource made available by the body issuing the clinical guidance. In some instances, the standards system 115 is a system associated with a provider or insurer that acts as a repository for clinical guidance as is it published. In an embodiment, the standards system 115 is configured to monitor and/or periodically request updates to clinical guidance data, as discussed in further detail below.

In various embodiments, the electronic network 130 is a wide area network (“WAN”), a local area network (“LAN”), personal area network (“PAN”), or the like. In some embodiments, electronic network 130 includes the Internet, and information and data provided between various systems occurs online. “Online” means connecting to or accessing source data or information from a location remote from other devices or networks coupled to the Internet. Alternatively, “online” refers to connecting or accessing an electronic network (wired or wireless) via a mobile communications network or device. The Internet is a worldwide system of computer networks-a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that includes data configured to cause a program such as a web browser to perform operations such as send, receive, or process data, generate a visual display and/or an interactive interface, or the like.

As discussed in further detail below, the training system 135, in embodiments, includes various programs, algorithms, or the like for performing various operations. While certain operations are described in association with one or more particular programs or algorithms, it should be understood that such operations may be distributed across any suitable number of algorithms or programs, and/or multiple operations may be performed by a single program or algorithm. In an example, the training system 135 includes one or more programs or algorithms for one or more of obtaining condition data, obtaining guidance data, parsing the condition data using the guidance data to generate training data, or training a machine-learning model.

As used herein, a “machine-learning model” generally encompasses instructions, data, and/or a model configured to receive input, and apply one or more of a weight, bias, classification, or analysis on the input to generate an output. The output includes, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model, in some cases, operate on an input linearly, in parallel, via a network (e.g., a neural network), or via any suitable configuration.

The execution of the machine-learning model, in some cases, includes deployment of one or more machine learning techniques, such as linear regression, logistical regression, random forest, gradient boosted machine (GBM), deep learning, and/or a deep neural network. Supervised and/or unsupervised training may be employed. For example, supervised learning sometimes includes providing training data and labels corresponding to the training data, e.g., as ground truth. Unsupervised approaches include clustering, classification or the like. K-means clustering or K-Nearest Neighbors is also usable, which may be supervised or unsupervised. Combinations of K-Nearest Neighbors and an unsupervised cluster technique are also usable. Any suitable type of training is usable, e.g., stochastic, gradient boosted, random seeded, recursive, epoch or batch-based, etc.

Generally, a machine-learning model includes a set of variables, e.g., nodes, neurons, filters, etc., that are tuned, e.g., weighted or biased, to different values via the application of training data. In supervised learning, e.g., where a ground truth is known for the training data provided, training proceeds by feeding a sample of training data into a model with variables set at initialized values, e.g., at random, based on Gaussian noise, a pre-trained model, or the like. The output is compared with the ground truth to determine an error, which is then back-propagated through the model to adjust the values of the variable.

Training may be conducted in any suitable manner, e.g., in batches, and includes any suitable training methodology, e.g., stochastic or non-stochastic gradient descent, gradient boosting, random forest, etc. In some embodiments, a portion of the training data is withheld during training and/or used to validate the trained machine-learning model, e.g., compare the output of the trained model with the ground truth for that portion of the training data to evaluate an accuracy of the trained model. The training of the machine-learning model, in some cases, is configured to cause the machine-learning model to learn associations between, for example, condition data and recommendations or decisions regarding a medical treatment, such that the trained machine-learning model is configured to determine a recommendation or decision for application of a treatment to a subject in response to the input of condition data for that subject, data based on the learned associations.

In various embodiments, the variables of a machine-learning model are interrelated in any suitable arrangement in order to generate the output. For example, in some embodiments, input data is arranged in a multi-dimensional array, and the machine-learning model includes architecture that is configured to identify, isolate, and/or extract features, geometry, and or structure in the array. For example, the machine-learning model includes one or more convolutional neural networks (“CNN”) configured to identify features in the condition data, and includes further architecture, e.g., a connected layer, neural network, etc., configured to determine a relationship between the identified features in order to generate a decision or recommendation.

In some instances, different samples or portions samples of training data and/or input data are not be independent. For example, a patient medical record may include information regarding a patient's condition over time. Thus, in some embodiments, the machine-learning model is configured to account for and/or determine relationships that occur over time or across different events. For example, in some embodiments, a machine-learning model includes a Recurrent Neural Network (“RNN”). Generally, RNNs are a class of feed-forward neural networks that are well adapted to processing a sequence of inputs. In some embodiments, the machine-learning model includes a Long Short Term Memory (“LSTM”) model and/or Sequence to Sequence (“Seq2Seq”) model. An LSTM model may be configured to generate an output from a sample that takes at least some previous samples and/or outputs into account. A Seq2Seq model may be configured to, for example, receive a sequence of non-optical in vivo images as input, and generate a sequence of locations, e.g., a path, in the medical imaging data as output. In another example embodiment, the machine-learning model includes a transformer architecture and/or an attention model, e.g., that includes or generates an encoding of where a portion of training sample is located relative to other data in the training sample.

In some embodiments, the training system 135 includes or has access to a database or data lake, such as storage of the provider system 110 configured to act as a repository or source for data such as patient medical records, or such as the standards system acting as a repository for guidance data. The training system 135, in various embodiments, includes one or more programs or algorithms configured to obtain data used for training a machine-learning model. For instance, in embodiments, the training system 135 is configured to obtain condition data, e.g., patient medical records for a plurality of subjects from the provider device 110 or the like. In different embodiments, such condition data is obtained periodically, in response to a user request, and/or in response to engaging in a training process for a machine-learning model. In some embodiments, the training system 135, when requesting condition data, identifies one or more characteristics of the condition data desired, e.g., a characteristic of the subject such as a particular condition or diagnosis, a decision made with regard to the subject such as a decision whether to apply a particular medical treatment or treatments, a particular window of time in which the data was generated such as within the last year or since a most-recent clinical guidance document was released, a particular provider or insurer or other grouping or categorization of subjects, etc.

In another example, the training system 135 includes a program or algorithm for obtaining criteria data, e.g., one or more clinical guidance documents from the standards system 115 or the like. In some embodiments, the training system includes a program or algorithm for extracting criteria from the criteria data, In an example, a keyword algorithm is configured to identify and extract keywords from criteria data, e.g., based on frequency of use, syntax, determined importance, etc., e.g., via natural language processing or the like. In another example, a summarization algorithm is configured to highlight topics or portions of content in criteria data, e.g., via natural language processing or the like. Any suitable filtering or highlighting technique for textual data or the like are usable.

In some embodiments, the training system 135 is configured to monitor a status of criteria data, e.g., on the standards system 115. In some instances, the training system 135 is configured to obtain updated criteria data, e.g., based on the monitoring, in response to a user request, and/or periodically. As discussed in further detail below, in some instances, the training system 135 is configured to automatically obtain relevant condition data and/or train or re-train a machine-learning model in response to receiving updated criteria data.

In a further example, the training system 135 includes an evaluation program or algorithm to generate one or more evaluations, such as a metric, question, sub-classification or the like, based on a portion of criteria data such as an extracted keyword, portion, topic, summary, or the like. For instance, the evaluation algorithm is configurable to identify a threshold or decision point in the criteria data based on extracted information. The evaluation algorithm is configurable, for example, to apply logical operators to aspects of the criteria data, e.g., relate portions or topics via an “if-then,” an “and,” an “or,” operator or the like. Application of such operators is based, in some embodiments, on natural language processing, semantic graphing or modelling, or any other suitable technique.

In another example, the training system 135 includes a natural language program or algorithm configured to generate natural language output for the metrics, questions, or the like generated by the evaluation algorithm. In an example, output generated by the natural language algorithm includes a distillation of criteria data down to one or more metrics or questions usable to evaluate whether a particular medical treatment corresponding to the criteria data should be applied to a subject. In an example, such output is in the form of a Q&A text interaction script, or the like.

The evaluation system 140 is configured to access and execute trained machine-learning models, e.g., to generate recommendations or decisions regarding potential application of a medical treatment to a subject 120. In some embodiments, the evaluation system 140 maintains a library of trained machine-learning models, e.g., different models that have been trained for different medical treatments. In an example, the models in the library may be indexed based on the classifications that the models are trained to generate. In some embodiments, the evaluation system 140 is configured to communicate with the training system 135, e.g., to request that a new machine-learning model for a particular medical treatment be configured and instantiated up (e.g., generated and trained). As discussed in further detail below, the evaluation system 140 is configured to interact with other systems or devices in the environment model to obtain data associated with using a machine-learning model, e.g., condition data for a target subject 120, criteria data, metrics, questions or the like associated with the machine-learning model, etc. In some embodiments, the evaluation system 140 includes or accesses a text generation program or algorithm configured to generate output text based on the recommendation or decision generated by the machine-learning algorithm and/or the associated metrics, questions or the like.

In an embodiment, the one or more criteria are extracted from the criteria or guidance data via a term frequency and/or inverse document frequency technique. In an embodiment, one or more pre-processing techniques may be applied to the criteria or guidance data, such as removal of stop words, identification of relationships or context between words, sentences, and/or paragraphs, or the like.

In an embodiment, the filtering of textual data, such as a patient record, based on guidance data includes any suitable matching process such as a string matching process, application of an algorithm or machine-learning model to assess word, sentence, and/or paragraph similarity. In an example, a Bidirectional Encoder Representations from Transformers (BERT) model is used to process words and/or sentences, and to generate distance measurements therebetween.

In an embodiment, the evaluation system 140 is configured to interact with or includes an interactive chat. In an example, a chat bot, or the like, prompts a user based on the one or more metrics or questions associated with a particular medical treatment. Prompts include, for example, a request for information from a subject's medical records and/or a result from evaluating the subject's medical records using the machine-learning model.

In an embodiment, the evaluation system 140 includes and/or is accessible via an API, e.g., such that another system such as a system of a provider, insurer, or the like is able to integrate decisions or recommendations of the evaluation system 140 into a workflow.

Although one or more examples above pertain to textual data, it should be understood that the techniques disclosed herein may be applied to any suitable type of data. For example, any type of data that may be encoded, e.g., into a vector or other format suitable as input for a machine-learning model, may be used such as image data, audio or video data, medical imaging data, three-dimensional model data, simulation data, etc. Moreover, while one or more of the examples above pertain to generating a decision or prediction for a preauthorization of a patient, it should be understood that any suitable prediction task may be the target classification of a machine-learning model according to one or more aspects of this disclosure.

Although depicted as separate components in FIG. 1, it should be understood that a component or portion of a component in the environment 100 is, in some embodiments, integrated with or incorporated into one or more other components. For example, a portion of the standards system 115 is integrated into the provider device 110, the training system 135, or the like. In another example, a portion of the training system 135 is integrated into the evaluation system 140, or vice versa. In a further example, one or more components are at least partially integrated into the user device 105. In some embodiments, operations or aspects of one or more of the components discussed above are distributed amongst one or more other components. Any suitable arrangement and/or integration of the various systems and devices of the environment 100 is usable in various embodiments. In one embodiment, one or more components of the environment are implemented as a cloud service or at least partially on a cloud environment, e.g., the standards system 115, the training system 135, the evaluation system 140, etc.

In the methods below, various acts are described as performed or executed by a component from the environment 100 of FIG. 1, such as the training system 135, the evaluation system 140, the provider device 110, the user device 105, or components thereof. However, it should be understood that in various embodiments, various components of the computing environment 100 discussed above execute instructions or perform acts including the acts discussed below. Moreover, an operation described as implemented by a particular component may also be understood to refer to execution of that operation by a corresponding program or algorithm, e.g., by one or more processors associated with that component or others. Further, it should be understood that in various embodiments, various steps can be added, omitted, and/or rearranged in any suitable manner.

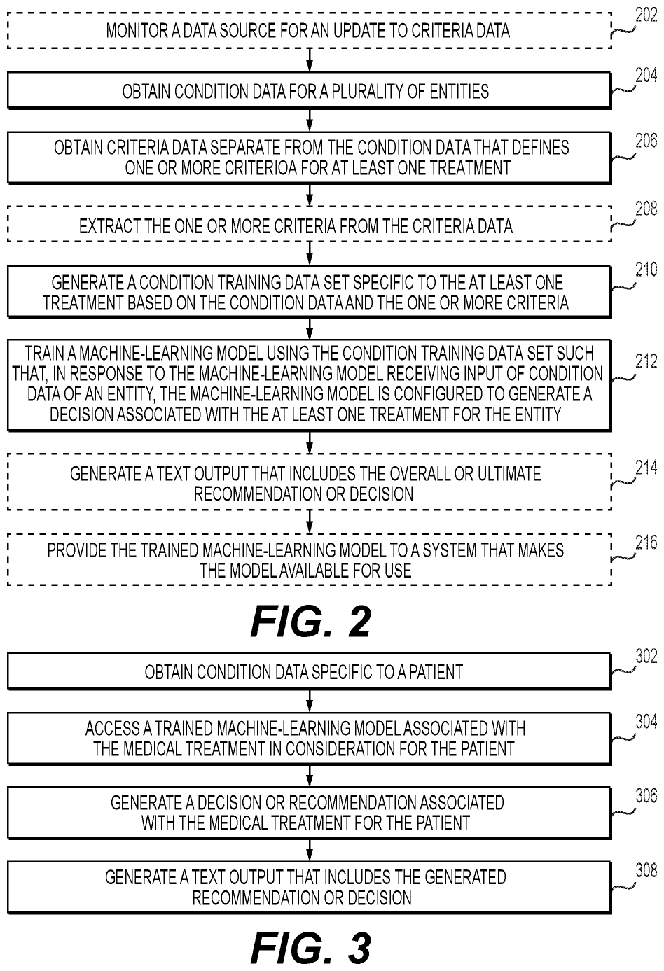

FIG. 2 illustrates an example process for training a machine-learning model, according to one or more aspects of this disclosure. In an example, a user, e.g., on behalf of an insurer, a provider, or the like, desires to obtain a recommendation or decision with regard to whether one or more medical treatments should be preauthorized and/or applied to one or more subjects, and engages a training system 135 to generate a machine-learning model trained to generate such output. In another example, the training system 135 is configured to automatically configure and instantiate new models. For instance, optionally, at step 202, the training system 135 monitors a data source, such as standards system 115 or the like, for an update to criteria data such as one or more clinical guidance documents. In some embodiments, the training system 135 is configured to obtain data and train a machine-learning model, such as via the following steps, in response to an update to available criteria data. In some instances, the monitoring is performed continuously, periodically, and/or in response to a request from the user.

At step 204, the training system 135 obtains condition data for a plurality of entities, e.g., patient medical records for a plurality of subjects 120 from the provider device 110 or the like. In some embodiments, the condition data includes corresponding prior decision data for application of one or more treatments to the plurality of subjects 120. For example, the condition data sometimes includes information regarding whether or not one or more medical treatments were applied to a subject 120, why or why not, and/or results thereof.

At step 206, the training system 135 obtains criteria data separate from the condition data that defines one or more criteria for at least one treatment, e.g., a clinical guidance document for one or more medical treatments from the standards system 115, or the like. The obtained criteria data, in some embodiments, is requested and/or selected based on a particular desired treatment or treatments. For instance, the machine-learning model may be configured and instantiated up to provide a recommendation or decision with regard to the particular desired treatment or treatments for a particular subject 120. In some embodiments, the obtained criteria data is updated criteria data obtained as a result of the monitoring in option al step 202.

In some embodiments, the criteria data includes one or more criteria for the corresponding treatment or treatments. In some embodiments, optionally at step 208, the training system 135 is configured to extract the one or more criteria from the criteria data. For example, in some embodiments, the training system 135 is configured to employ one or more algorithms or programs to extract or identify one or more keywords, portions, topics, or summaries from the criteria data, and/or employ one or more algorithms or programs to generate one or more metrics, questions, or the like based on such information. In an example, a similarity-based matching algorithm or the like may be applied, such as a cosine similarity algorithm. Such metrics, questions, or the like, in various embodiments, have any suitable logical relationship.

At step 210, the training system 135 generates a condition training data set specific to the at least one treatment based on the condition data and the one or more criteria. In an example, generating the condition training data set includes filtering the condition data, based on the one or more criteria. In an example, for each sample of condition data, the training system 135 identifies a respective portion corresponding to each keyword, portion, topic, or summary from the criteria data. In another example, for each metric, question, or the like, the training system 135 identifies a respective portion of each sample. The identified portions of each sample are extracted to, in each case, form a sample of the condition training data set specific to the at least one treatment. In an example use case, according to one or more aspects of this disclosure, information from each sample that is relevant to the particular medical treatment or treatments at issue and/or the metrics, questions or the like that are generated for the particular medical treatment or treatments is extracted from the condition data, e.g., to filter out from the condition data information that is less relevant to the evaluation of the particular medical treatment or treatments.

In some embodiments, the samples of the condition training data set specific to the at least one treatment are unlabeled, e.g., are not associated with an outcome or decision for the subjects corresponding to the underlying condition data. In some embodiments, the samples of the condition training data set specific to the at least one treatment are labeled based on the corresponding prior decision data. For example, in some embodiments, the training system 135 associates each sample with information regarding an outcome or decision on the particular medical treatment or treatments for each subject. In some embodiments, the condition data includes information regarding such out outcome or decision, and so is included in the condition training data set during the filtering process or at any other suitable step.

At step 212, the training system 135 trains a machine-learning model using the condition training data set such that, in response to the machine-learning model receiving input of medical data of an entity, the machine-learning model is configured to generate a decision associated with the at least one treatment for the entity. Any suitable machine-learning training technique is usable, such as one or more of the techniques discussed above. In some embodiments, training the machine-learning model includes configuring the machine-learning model to develop a respective metric for each of the one or more criteria, such that the decision generated by the machine-learning model includes a separate evaluation of each metric.

For example, in an embodiment in which one or more criteria such as a metric, question, or the like is identified at step 208, the machine-learning model is configured to predict an outcome for the criterion based on the condition training data set. In one embodiment, the one or more criteria are included and/or injected into a layer of a deep learning network prior to a final fulling connected layer corresponding to the ultimate prediction or decision generated by the model. In another embodiment, a separate sub-machine-learning model is generated and trained for each criterion, the output for which is fed as input to a further machine-learning model to generate the ultimate recommendation or decision. Any suitable technique for incorporating discrete criteria into the machine-learning model is usable in various embodiments. In such embodiments, e.g., in which training the machine-learning model includes configuring the machine-learning model to develop a respective metric for each of the one or more criteria, the decision generated by the machine-learning model or models thus includes a separate evaluation of each metric.

In an example use case, each of a plurality of different medical treatments is to be evaluated. Clinical guidance data for the plurality of different medical treatments is obtained, e.g., as set forth in one or more of the examples above. The training system 135, e.g., when employing the evaluation algorithm discussed above, determines that one or more of the plurality of different medical treatments are logically separate, e.g., capable of being evaluated separately. Each such treatment is thus identified as a separate criterion or metric. As a result, the machine-learning model, once trained such as via one or more of the examples above, is trained to generate a respective decision or recommendation for each of the plurality of treatments. In some embodiments, one treatment is related to another, e.g., sequentially, (one is a prerequisite to another), hierarchically (one is a sub-step of another), or negatively (one is not performed if another is), and/or by any other suitable logical relationship.

Optionally, at step 214, the training system 135 is configured to generate a text output that includes the overall or ultimate recommendation or decision. In some embodiments, the text output further includes each metric. In some embodiments, the text output further includes an impact or weight that each metric had on the overall outcome.

Optionally, at step 216, the training system 135 is configured to provide the trained machine-learning model to a system that makes the model available for use, e.g., a library of models associated with the evaluation system 140. As discussed in further detail below, such a library is configurable to host a plurality of trained machine-learning models for different medical treatments, and is configurable to provide access to a selection of one or more trained models corresponding to receiving an identification of one or more treatments.

In an example embodiment applying one or more aspects of the method of FIG. 2 discussed above, a machine-learning model is trained to generate recommendations and/or decisions for a plurality of different medical treatments. In this example, at step 204, the condition data obtained by the training system 135 includes condition data of a plurality of different subjects 120 that are being considered for and/or that received one of a plurality of different medical treatments. Further, at step 206, the criteria data obtained by the training system 135 includes different criteria data for each of the plurality of different medical treatments. Continuing, at step 210 for the example, the training system 135 generates the condition training data set by, in each case, appending at least a portion of the respective criteria data to each sample of condition data related to a corresponding medical treatment. In some embodiments, the appended portion of criteria data is a portion resulting from a filtering process such as via one or more of the examples discussed above. In an example use case, each sample of the condition training data set is an array that includes the condition data of a subject 120 appended with a portion of criteria data corresponding to a medical treatment considered for or applied to the subject 120. A machine-learning model is then trained using the condition training data set, as per step 212. In this manner, the machine-learning model is trained to receive input that, in each instance, includes not only condition data of a subject 120, but also criteria data for a medical treatment specific to that subject 120. In some instances, the trained machine-learning model of the foregoing example acts as a generalized model capable of receiving criteria data for a medical treatment that was not included in the training data, e.g., as a zero-shot model. In some instances, the trained-machine learning model according to the foregoing example, is able to receive and apply criteria data that has been updated relative to criteria data used to train the model, e.g., so that the model need not be re-trained in order to account for updates to criteria data.

It should be understood that the operations above for training a machine-learning model are illustrative only, and that any suitable techniques may be used. Further, although examples above pertain to generating recommendations or decisions for preauthorization or application of a medical treatment to a subject 120, it should be understood that one or more aspects of the foregoing is applicable to any suitable activity.

FIG. 3 depicts an example embodiment of a process for generating a recommendation or decision associated with at least one treatment for an entity, e.g., a recommendation or decision regarding preauthorization or application of a medical treatment or treatments to a subject 120, according to one or more aspects of this disclosure. One or more medical treatments may be considered for a subject 120, e.g., a patient of a provider. At step 302, condition data specific to the patient, e.g., a medical record of the patient that includes medical history data, test results, observations, vital statistics, demographic information, environmental information, etc., is obtained. The patient-specific condition data includes an identification of the medical treatment in consideration for the patient.

In an example, a user obtains the patient-specific condition data using a user device 105 that is in communication with one or more of the provider device 110, or the like, e.g., via an API, and such condition data is passed to the evaluation system 140. In another example, the patient-specific condition data is obtained at least partially automatically, e.g., via the evaluation system 140. Such operation of the evaluation system 140 is performed, in different embodiments, in response to a user request, via an automatic process of the provider device 110, via an automatic process of the evaluation system 140, etc. For instance, in an example use case, once a medical treatment is entered for consideration of a patient, the provider device 110 is configured to interact with the evaluation system 140 for generating a recommendation or decision, and the evaluation system 140 is configured to automatically request and/or obtain the patient-specific condition data in response.

At step 304, the evaluation system 140, e.g., in response to a user request and/or in response to obtaining the patient-specific condition data, accesses a trained machine-learning model associated with the medical treatment in consideration for the patient. In an example, the evaluation system 140 accesses a storage that includes one or more machine-learning models, such as the library discussed above, and accesses a corresponding model.

At step 306, the evaluation system 140 generates, e.g., using the accessed machine-learning model, a decision or recommendation associated with the medical treatment for the patient. In an example, the generating includes inputting the patient-specific condition data into the accessed machine-learning model, which has been trained, e.g., via one or more aspects of the method of FIG. 2 discussed above, based on a condition training data set. As discussed above, the condition training data stet has been generated by using one or more criteria to filter condition data for a plurality of patients. In some embodiments, the decision or recommendation generated by the machine-learning model includes a separate evaluation of one or more metrics corresponding to the one or more criteria, e.g., as discussed in one or more of the examples above.

At step 308, the evaluation system 140 generates a text output that includes the generated recommendation or decision. In some embodiments, the text output is appended to the condition data of the patient. In some embodiments, the evaluation system 140 causes the user device 105 to output the text output. In some embodiments, the text output further includes the one or more metrics and the separate evaluation of the one or more metrics.

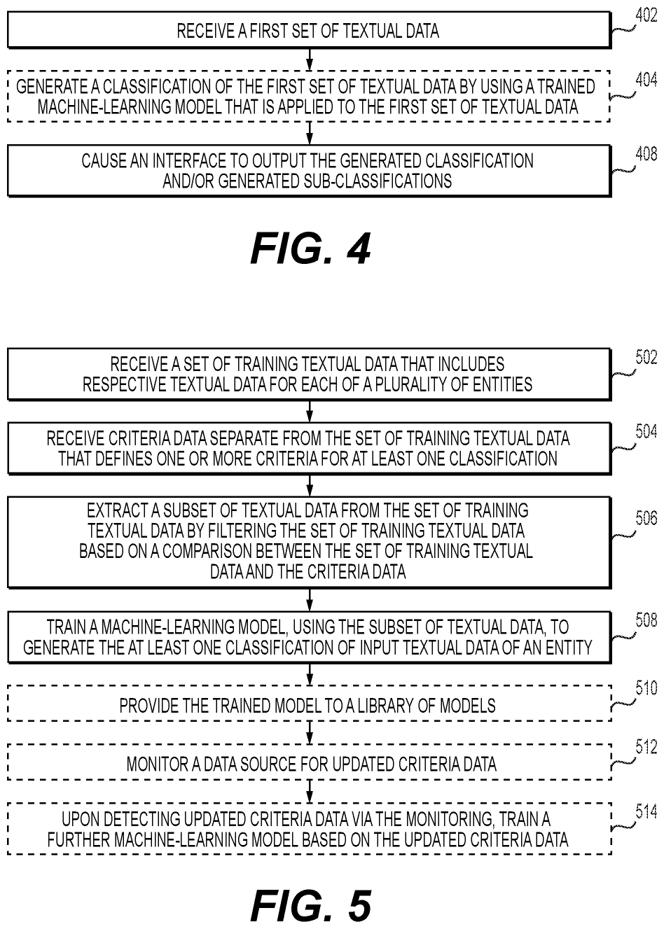

FIG. 4 depicts another example embodiment of using a machine-learning model, according to one or more aspects of this disclosure. Obtaining a classification or prediction of textual data may be desired. At step 402, a system, e.g., an evaluation system 140, receives a first set of textual data, e.g., a patient record for a particular patient 120. In an example, a user such as a provider 125 may input the first set of textual data into the evaluation system 140, e.g., via an API, a chat bot, or any other suitable interface. In another example, the evaluation system 140 automatically obtains the first set of textual data. In some instances, the first set of textual data includes one or more coded entries, e.g., portions of the textual data that are associated with or categorized by a respective code such as a medical code, or the like.

At step 404, a classification of the first set of textual data is generated by using a trained machine-learning model that is applied to the first set of textual data. In an example, the trained machine-learning model has been trained based on a subset of textual data that resulted from filtering a set of training textual data. The set of training textual data includes, for example, patient records from a plurality of patients that previously received preauthorization decisions, classifications, treatment decisions, treatments, or the like. In some instances, the set of training textual data includes prior classification or decision data, e.g., a prior decision applied to the patient record of each of the plurality of patients in the set of training textual data. In some instances, the set of training textual data includes one or more coded entries, e.g., portions of the textual data that are associated with or categorized by a respective code such as a medical code, or the like.

In an example, filtering of the set of training textual data to generate the subset of textual data is based on a comparison between the training textual data and a second set of textual data. The second set of textual data includes, for example, criteria or guidance data, e.g., medical guidelines associated with a medical treatment. In an example, the comparison is based on a comparison of codes associated with coded entries of the training textual data and of the second set of textual data.

In an example, the filtering includes applying a similarity-based matching technique to the set of training textual data and the second set of textual data. Any suitable matching technique may be used, such as a cosine similarity or the like.

In another example, the filtering includes extracting one or more keywords from the second set of textual data, and then extracting, as the subset of textual data, one or more portions of the set of training textual data corresponding to the one or more keywords.

In an example, one or more sub-classification metrics were determined based on the set of training textual data and/or the second set of textual data. In an example, the machine-learning model was further trained using the one or more sub-classification metrics such that the trained machine-learning model is configured to generate a respective sub-classification of the first set of textual data for each of the one or more sub-classification metrics. In an example, the trained machine-learning model is further configured to generate the classification of the first set of textual data based on the one or more respective sub-classifications. In some instances, the output of the trained machine-learning model includes a textual representation of the one or more respective sub-classifications, e.g., in a Q&A format.

Optionally, at step 406, the evaluation system 140 causes the interface to output the generated classification and/or sub-classifications. For instance, the output may be via the chat bot, via a visual display, or via any other suitable technique.

FIG. 5 depicts another example embodiment of training a machine-learning model, according to one or more aspects of this disclosure. At step 502, a system, e.g., the training system 135, receives a set of training textual data that includes respective textual data for each of a plurality of entities. For example, the training system 135 may receive a set of patient records as discussed in one or more of the examples above.

At step 504, the training system 135 receives criteria data separate from the set of training textual data that defines one or more criteria for at least one classification. The criteria data may include, for example, medical guidance for performance of a medical procedure as discussed in one or more of the examples above.

At step 506, the training system 135 extracts a subset of textual data from the set of training textual data by filtering the set of training textual data based on a comparison between the set of training textual data and the criteria data. The filtering, in various embodiments, includes one or more aspects of filtering discussed in the one or more examples above. For example, the training system 135, in an embodiment, extracts one or more criteria, classification, sub-classification, or the like from the criteria data.

At step 508, the training system 135 trains a machine-learning model, using the subset of textual data, to generate the at least one classification of input textual data of an entity. In other words, once trained, the machine-learning model is configured to classify input textual data, e.g., generate or predict a preauthorization decision of a particular medical treatment for a patient in response to input of the patient's medical record. Any suitable training procedure is usable. In an example, the machine-learning model is trained using one or more sub-classifications extracted as discussed above, such that the training causes the machine-learning model to develop a respective sub-classification for each of the one or more criteria defined by the criteria data.

In an example, different machine-learning models are trained with different sets of training data and different guidance data. In an example, a particular machine-learning model is trained using a set of patient records from patients that exhibited a particular condition and a guidance document associated with one or more treatments for that particular condition.

Optionally, at step 510, the training system 135 may provide the trained model to a library of models. The library, for example, is configured to index models based on classification. In other words, in an example, the library is configured to provide access to a particular model in response to identification of a desired classification. For instance, a provider 125 may desire to obtain a prediction, recommendation, decision, classification, or the like for a patient 120 that has a particular condition. The provider 125, via evaluation system 140, may access the library to obtain a trained model corresponding to that particular condition.

Optionally, at step 512, the evaluation system 140 monitors a data source such as the standards system 115 for updated criteria data. Optionally, at step 514, upon detecting updated criteria data via the monitoring, the evaluation system 140 trains a further machine-learning model based on the updated criteria data. In an example, the further machine-learning model replaces the trained machine-learning model from step 508. In an example, the training of the further machine-learning model uses a set of training textual data for which the updated criteria data was applied. For instance, the further machine-learning model is trained using patient data only from patients that were evaluated using current medical guidelines, e.g., so that the further machine-learning model is not trained to apply outdated guidelines.

It should be understood that embodiments in this disclosure are examples only, and that other embodiments may include various combinations of features from other embodiments, as well as additional or fewer features. For example, while some of the embodiments above pertain to a sequential randomized trial operated in conjunction with a distributed messaging platform, any suitable sequential randomized trial with any suitable type or kind of treatment may be implemented according to one or more aspects of this disclosure.

In general, any process or operation discussed in this disclosure that is understood to be computer-implementable, such as the processes illustrated in FIGS. 2 and 3, is performed by one or more processors of a computer system, such any of the systems or devices in the environment 100 of FIG. 1, as described above. A process or process step performed by one or more processors may also be referred to as an operation. The one or more processors are configured to perform such processes by having access to instructions (e.g., software or computer-readable code) that, when executed by the one or more processors, cause the one or more processors to perform the processes. The instructions are stored in a memory of the computer system. A processor is a central processing unit (CPU), a graphics processing unit (GPU), or any suitable types of processing unit.

A computer system, such as a system or device implementing a process or operation in the examples above, includes one or more computing devices, such as one or more of the systems or devices in FIG. 1. One or more processors of a computer system are included in a single computing device or distributed among a plurality of computing devices. A memory of the computer system includes the respective memory of each computing device of the plurality of computing devices.

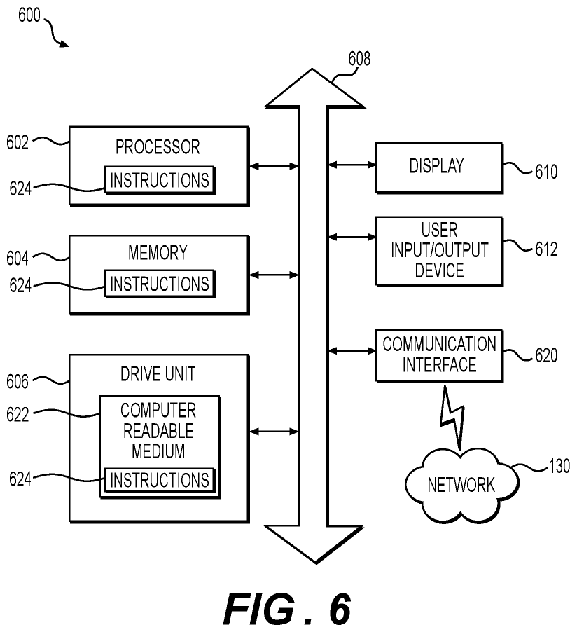

FIG. 6 is a simplified functional block diagram of a computer 600 that is configured as a device for executing the methods of FIGS. 2 and 3, according to example embodiments of the present disclosure. For example, the computer 600 is configured as the training system 135, evaluation system 140, and/or another system according to example embodiments of this disclosure. In various embodiments, any of the systems herein is a computer 600 including, for example, a data communication interface 620 for packet data communication. The computer 600 also includes a central processing unit (“CPU”) 602, in the form of one or more processors, for executing program instructions. The computer 600 includes an internal communication bus 608, and a storage unit 606 (such as ROM, HDD, SDD, etc.) that stores data on a computer readable medium 622, although the computer 600 receives programming and data via network communications. The computer 600, in embodiments, also has a memory 604 (such as RAM) storing instructions 624 for executing techniques presented herein, although the instructions 624 are stored temporarily or permanently within other programs of computer 600 (e.g., processor 602 and/or computer readable medium 622). The computer 600 also includes input and output ports 612 and/or a display 610 to connect with input and output devices such as keyboards, mice, touchscreens, monitors, displays, etc. The various system functions are implemented in a distributed fashion on a number of similar platforms, to distribute the processing load. Alternatively, the systems is implemented by appropriate programming of one computer hardware platform.

Program aspects of the technology are thought of as “products” or “articles of manufacture” typically in the form of executable code and/or associated data that is carried on or embodied in a type of machine-readable medium. “Storage” type media include any or all of the tangible memory of the computers, processors or the like, or associated programs thereof, such as various semiconductor memories, tape drives, disk drives and the like, which is able to provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that stores the software elements includes optical, electrical and electromagnetic waves, such as used across physical interfaces between local devices, through wired and optical landline networks and over various air-links. The physical elements that carry such waves, such as wired or wireless links, optical links, or the like, also is considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with illustrative reference to transmitting data, it should be appreciated that the disclosed embodiments are applicable to any environment, such as a desktop or laptop computer, an automobile entertainment system, a home entertainment system, etc. Also, the disclosed embodiments are applicable to any type of Internet protocol.

It should be appreciated that in the above description of example embodiments of the present disclosure, various features of the present disclosure are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed present disclosure requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this present disclosure.

Furthermore, while some embodiments described herein include some but not other features included in other embodiments, combinations of features of different embodiments are meant to be within the scope of the present disclosure, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the present disclosure, and it is intended to claim all such changes and modifications as falling within the scope of the present disclosure. For example, functionality may be added or deleted from the block diagrams and operations may be interchanged among functional blocks. Steps may be added or deleted to methods described within the scope of the present disclosure.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

The present disclosure furthermore relates to the following aspects.

Example 1. A computer-implemented method for training a machine-learning model, comprising: obtaining, via one or more processors, condition data for a plurality of entities; obtaining, via the one or more processors, criteria data separate from the condition data that defines one or more criteria for at least one treatment; filtering the condition data, via the one or more processors and based on the one or more criteria, to generate a condition data training set specific to the at least one treatment; and training a machine-learning model, via the one or more processors and using the condition data training set, such that, in response to the machine-learning model receiving input of medical data of an entity, the machine-learning model is configured to generate a decision associated with the at least one treatment for the entity.

Example 2. The computer-implemented method of example 1, further comprising: extracting, via the one or more processors, the one or more criteria from the criteria data.

Example 3. The computer-implemented method of any preceding example, wherein obtaining the criteria data includes monitoring a data source for an update to the criteria data, such that the machine-learning model is successively trained with reference to updated criteria data in response to each update.

Example 4. The computer-implemented method of any preceding example, wherein: the condition data includes corresponding prior decision data for application of the treatment to the plurality of entities; and the condition data training set is labeled based on the corresponding prior decision data.

Example 5. The computer-implemented method of any preceding example, wherein the condition data training set consists of data that is not labeled.

Example 6. The computer-implemented method of any preceding example, wherein training the machine-learning model includes configuring the machine-learning model to develop a respective metric for each of the one or more criteria, such that the decision generated by the machine-learning model includes a separate evaluation of each metric.

Example 7. The computer-implemented method of example 6, wherein the machine-learning model is further configured to generate a text output that includes each metric and an impact on the overall decision by each metric.

Example 8. The computer-implemented method of any preceding example, wherein the criteria data includes one or more criteria for each of a plurality of treatments, such that the machine-learning model is trained to generate a respective decision for each of the plurality of treatments.

Example 9. The computer-implemented method of example 8, further comprising: providing the trained machine-learning model to a library of models for different treatments, the library configured to provide access to a selection of one or more trained models corresponding to receiving an identification of one or more treatments.

Example 10. A system for training a machine-learning model, comprising: at least one memory storing instructions; and at least one processor operatively connected to the at least one memory and configured to execute the instructions to perform operations, including: obtaining condition data for a plurality of entities; obtaining criteria data separate from the condition data that defines one or more criteria for at least one treatment; filtering the condition data, based on the one or more criteria, to generate a condition data training set specific to the at least one treatment; and training a machine-learning model, using the condition data training set, such that, in response to the machine-learning model receiving input of medical data of an entity, the machine-learning model is configured to generate a decision associated with the at least one treatment for the entity.

Example 11. The system of example 10, wherein: obtaining the criteria data includes monitoring a data source for an update to the criteria data, such that the machine-learning model is successively trained with reference to updated criteria data in response to each update; and the operations further include extracting the one or more criteria from the criteria data.

Example 12. The system of any of examples 10-11, wherein: the condition data includes corresponding prior decision data for application of the treatment to the plurality of entities; and the condition data training set is labeled based on the corresponding prior decision data.

Example 13. The system of any of examples 10-12, wherein the condition data training set consists of data that is not labeled.

Example 14. The system of any of examples 10-13, wherein: training the machine-learning model includes configuring the machine-learning model to develop a respective metric for each of the one or more criteria, such that the decision generated by the machine-learning model includes a separate evaluation of each metric; and the machine-learning model is further configured to generate a text output that includes each metric and an impact on the overall decision by each metric.

Example 15. The system of any of examples 10-14, wherein: the criteria data includes one or more criteria for each of a plurality of treatments, such that the machine-learning model is trained to generate a respective decision for each of the plurality of treatments; and the operations further include providing the trained machine-learning model to a library of models for different treatments, the library configured to provide access to a selection of one or more trained models corresponding to receiving an identification of one or more treatments.

Example 16. A non-transitory computer-readable medium comprising instructions for training a machine-learning model, the instructions executable by at least one processor to perform operations, including: obtaining condition data for a plurality of entities; obtaining criteria data separate from the condition data that defines one or more criteria for at least one treatment; filtering the condition data, based on the one or more criteria, to generate a condition data training set specific to the at least one treatment; and training a machine-learning model, using the condition data training set, such that, in response to the machine-learning model receiving input of medical data of an entity, the machine-learning model is configured to generate a decision associated with the at least one treatment for the entity.

Example 17. The non-transitory computer-readable medium of example 16, wherein: obtaining the criteria data includes monitoring a data source for an update to the criteria data, such that the machine-learning model is successively trained with reference to updated criteria data in response to each update; and the operations further include extracting the one or more criteria from the criteria data.

Example 18. The non-transitory computer-readable medium of any of examples 16-17, wherein the condition data includes corresponding prior decision data for application of the treatment to the plurality of entities.

Example 19. The non-transitory computer-readable medium of any of examples 16-18, wherein: training the machine-learning model includes configuring the machine-learning model to develop a respective metric for each of the one or more criteria, such that the decision generated by the machine-learning model includes a separate evaluation of each metric; and the machine-learning model is further configured to generate a text output that includes each metric, the separate evaluation of each metric, and an overall decision based on a combined evaluation of each metric.

Example 20. The non-transitory computer-readable medium of any of examples 16-19, wherein: the criteria data includes one or more criteria for each of a plurality of treatments, such that the machine-learning model is trained to generate a respective decision for each of the plurality of treatments; and the operations further include providing the trained machine-learning model to a library of models for different treatments, the library configured to provide access to a selection of one or more trained models corresponding to receiving an identification of one or more treatments.

Example 21. A computer-implemented method for generating a decision associated with at least one treatment for an entity, comprising: obtaining, via at least one processor of a user device, entity-specific condition data; generating, via the at least one processor, the decision associated with the at least one treatment for the entity by inputting the entity-specific condition data into a trained machine-learning model that has been trained, based on a condition data training set generated by using one or more criteria to filter condition data for a plurality of entities, wherein the decision generated by the trained machine-learning model includes a separate evaluation of one or more metrics corresponding to the one or more criteria; and generating a text output that includes the one or more metrics, the separate evaluation of the one or more metrics, and an overall decision based on a combined evaluation of each metric.

Example 22. A computer-implemented method, comprising: receiving, via one or more processors, a first set of textual data; and generating, via the one or more processors and using a trained machine-learning model that is applied to the first set of textual data, a classification of the first set of textual data, wherein: the trained machine-learning model has been trained based on a subset of textual data that resulted from filtering a set of training textual data; and the filtering of the set of training textual data to generate the subset of textual data is based on a comparison between the training textual data and a second set of textual data.

Example 23. The computer-implemented method of example 22, wherein the filtering includes: extracting one or more keywords from the second set of textual data; and extracting, as the subset of textual data, one or more portions of the set of training textual data corresponding to the one or more keywords.

Example 24. The computer-implemented method of any of examples 22-23, wherein the filtering includes applying a similarity-based matching technique to the set of training textual data and the second set of textual data.

Example 25. The computer-implemented method of example 24, wherein the similarity-based matching technique includes a cosine similarity.

Example 26. The computer-implemented method of any of example 22-25, wherein the set of training textual data includes one or more prior classifications applied to the training textual data.

Example 27. The computer-implemented method of any of examples 22-26, wherein: the first set of textual data is received via an interactive chat with a chat bot; and the method further comprises causing the chat bot to output the generated classification of the first set of textual data.

Example 28. The computer-implemented method of any of examples 22-27, wherein the set of training textual data includes one or more coded entries that are each associated with or categorized by a respective code.

Example 29. The computer-implemented method of any of examples 22-28, wherein: the trained machine-learning model was further trained using one or more sub-classification metrics determined based on the set of training textual data, such that the trained machine-learning model is configured to generate a respective sub-classification of the first set of textual data for each of the one or more sub-classification metrics; and the trained machine-learning model is further configured to generate the classification of the first set of textual data based on the one or more respective sub-classifications.

Example 30. A system, comprising: at least one memory storing instructions; and at least one processor operatively connected to the at least one memory and configured to execute the instructions to perform operations, including: receiving a first set of textual data; and generating, using a trained machine-learning model that is applied to the first set of textual data, a classification of the first set of textual data, wherein: the trained machine-learning model has been trained based on a subset of textual data that resulted from filtering a set of training textual data; and the filtering of the set of training textual data to generate the subset of textual data is based on a comparison between the training textual data and a second set of textual data.

Example 31. The system of example 30, wherein the filtering includes: extracting one or more keywords from the second set of textual data; and extracting, as the subset of textual data, one or more portions of the set of training textual data corresponding to the one or more keywords.

Example 32. The system of any of examples 30-31, wherein the filtering includes applying a similarity-based matching technique to the set of training textual data and the second set of textual data.

Example 33. The system of any of examples 30-32, wherein the set of training textual data includes at least one of: one or more prior classifications applied to the training textual data; or one or more coded entries that are each associated with or categorized by a respective code.

Example 34. The system of any of examples 30-33, wherein: the first set of textual data is received via an interactive chat with a chat bot; and the operations further include causing the chat bot to output the generated classification of the first set of textual data.

Example 35. The system of any of examples 30-34, wherein: the trained machine-learning model was further trained using one or more sub-classification metrics determined based on the set of training textual data, such that the trained machine-learning model is configured to generate a respective sub-classification of the first set of textual data for each of the one or more sub-classification metrics; and the trained machine-learning model is further configured to generate the classification of the first set of textual data based on the one or more respective sub-classifications.

Example 36. A computer-implemented method, comprising: receiving, via one or more processors, a set of training textual data that includes respective textual data for each of a plurality of entities; receiving, via the one or more processors, criteria data separate from the set of training textual data that defines one or more criteria for at least one classification; extracting a subset of textual data from the set of training textual data by filtering the set of training textual data based on a comparison between the set of training textual data and the criteria data; and training a machine-learning model, via the one or more processors and using the subset of textual data, to generate the at least one classification of input textual data of an entity.

Example 37. The computer-implemented method of example 36, further comprising: extracting, via the one or more processors, the one or more criteria from the criteria data.

Example 38. The computer-implemented method of any of examples 36-37, further comprising: monitoring, via the one or more processors, a data source for updated criteria data; and upon detecting the updated criteria data via the monitoring, training a further machine-learning model based on the updated criteria data.

Example 39. The computer-implemented method of any of examples 36-38, wherein the criteria data is labeled based on respective prior classifications of the respective textual data for each of a plurality of entities.

Example 40. The computer-implemented method of any of examples 36-39, wherein: training the machine-learning model includes causing the machine-learning model to develop a respective sub-classification for each of the one or more criteria defined by the criteria data; and the classification that the machine-learning model is trained to generate includes the respective sub-classification for each of the one or more criteria.

Example 41. The computer-implemented method of any of examples 36-40, further comprising: after the training, providing the machine-learning model to a library of models indexed based on classification, the library configured to provide access to a particular model in response to identification of a desired classification.

What is claimed is:

1. A computer-implemented method, comprising:
receiving, via one or more processors, a first set of textual data; and
generating, via the one or more processors and using a trained machine-learning model that is applied to the first set of textual data, a classification of the first set of textual data, wherein:
the trained machine-learning model has been trained based on a subset of textual data that resulted from filtering a set of training textual data; and
the filtering of the set of training textual data to generate the subset of textual data is based on a comparison between the training textual data and a second set of textual data.

2. The computer-implemented method of claim 1, wherein the filtering includes:
extracting one or more keywords from the second set of textual data; and
extracting, as the subset of textual data, one or more portions of the set of training textual data corresponding to the one or more keywords.

3. The computer-implemented method of claim 1, wherein the filtering includes applying a similarity-based matching technique to the set of training textual data and the second set of textual data.

4. The computer-implemented method of claim 3, wherein the similarity-based matching technique includes a cosine similarity.

5. The computer-implemented method of claim 1, wherein the set of training textual data includes one or more prior classifications applied to the training textual data.

6. The computer-implemented method of claim 1, wherein:
the first set of textual data is received via an interactive chat with a chat bot; and
the method further comprises causing the chat bot to output the generated classification of the first set of textual data.

7. The computer-implemented method of claim 1, wherein the set of training textual data includes one or more coded entries that are each associated with or categorized by a respective code.

8. The computer-implemented method of claim 1, wherein:
the trained machine-learning model was further trained using one or more sub-classification metrics determined based on the set of training textual data, such that the trained machine-learning model is configured to generate a respective sub-classification of the first set of textual data for each of the one or more sub-classification metrics; and
the trained machine-learning model is further configured to generate the classification of the first set of textual data based on the one or more respective sub-classifications.

9. A system, comprising:
at least one memory storing instructions; and
at least one processor operatively connected to the at least one memory and configured to execute the instructions to perform operations, including:
receiving a first set of textual data; and
generating, using a trained machine-learning model that is applied to the first set of textual data, a classification of the first set of textual data, wherein:
the trained machine-learning model has been trained based on a subset of textual data that resulted from filtering a set of training textual data; and
the filtering of the set of training textual data to generate the subset of textual data is based on a comparison between the training textual data and a second set of textual data.

10. The system of claim 9, wherein the filtering includes:
extracting one or more keywords from the second set of textual data; and
extracting, as the subset of textual data, one or more portions of the set of training textual data corresponding to the one or more keywords.

11. The system of claim 9, wherein the filtering includes applying a similarity-based matching technique to the set of training textual data and the second set of textual data.

12. The system of claim 9, wherein the set of training textual data includes at least one of:
one or more prior classifications applied to the training textual data; or
one or more coded entries that are each associated with or categorized by a respective code.

13. The system of claim 9, wherein:
the classification is a domain-specific classification; and
the second set of textual data includes domain-specific information associated with the domain-specific classification.

14. The system of claim 9, wherein:
the trained machine-learning model was further trained using one or more sub-classification metrics determined based on the set of training textual data, such that the trained machine-learning model is configured to generate a respective sub-classification of the first set of textual data for each of the one or more sub-classification metrics; and
the trained machine-learning model is further configured to generate the classification of the first set of textual data based on the one or more respective sub-classifications.

15. A computer-implemented method, comprising:
receiving, via one or more processors, a set of training textual data that includes respective textual data for each of a plurality of entities;
receiving, via the one or more processors, criteria data separate from the set of training textual data that defines one or more criteria for at least one classification;
extracting a subset of textual data from the set of training textual data by filtering the set of training textual data based on a comparison between the set of training textual data and the criteria data; and
training a machine-learning model, via the one or more processors and using the subset of textual data, to generate the at least one classification of input textual data of an entity.

16. The computer-implemented method of claim 15, further comprising:
extracting, via the one or more processors, the one or more criteria from the criteria data.

17. The computer-implemented method of claim 15, further comprising:
monitoring, via the one or more processors, a data source for updated criteria data; and
upon detecting the updated criteria data via the monitoring, training a further machine-learning model based on the updated criteria data.

18. The computer-implemented method of claim 15, wherein the criteria data is labeled based on respective prior classifications of the respective textual data for each of a plurality of entities.

19. The computer-implemented method of claim 15, wherein:
training the machine-learning model includes causing the machine-learning model to develop a respective sub-classification for each of the one or more criteria defined by the criteria data; and
the classification that the machine-learning model is trained to generate includes the respective sub-classification for each of the one or more criteria.

20. The computer-implemented method of claim 15, further comprising:
after the training, providing the machine-learning model to a library of models indexed based on classification, the library configured to provide access to a particular model in response to identification of a desired classification.
